(19) United States
(12) Patent Application Publication
(10) Pub. No.: US 2024/0355435 A1
(43) Pub. Date: Oct. 24, 2024

(54) A METHOD USED FOR DETERMINING PERSONAL REFERENCE RANGE OF MEDICAL LABORATORY TESTS

(71) Applicant: ACIBADEM MEHMET ALI AYDINLAR UNIVERSITESI, Istanbul (TR)

(72) Inventors: Abdurrahman COSKUN, Istanbul (TR); Sverre SANDBERG, Bergen (NO); Ibrahim UNSAL, Istanbul (TR); Coskun CAVUSOGLU, Istanbul (TR); Mustafa SERTESER, Istanbul (TR); Meltem KILERCIK, Istanbul (TR); Aasne Karine AARSAND, Bergen (TR)

(73) Assignee: ACIBADEM MEHMET ALI AYDINLAR UNIVERSITESI, Istanbul (TR)

(21) Appl. No.: 18/294,241

(22) PCT Filed: Dec. 28, 2021
(86) PCT No.: PCT/TR2021/051546

§ 371 Date: Feb. 1, 2024

(30) Foreign Application Priority Data
Aug. 2, 2021 (TR) 2021/012216

(51) Int. Cl. G16H 10/40; G16H 50/30

(52) U.S. Cl. CPC G16H 10/40; G16H 50/30

(57) ABSTRACT

The present invention relates to a method (100) which enables to calculate a personal reference range in order to compare results of measurements that are carried out in case of suspicion of disease by using the results of laboratory tests measured in a period when persons feel healthy.

### TECHNICAL FIELD

The present invention relates to a method for calculating a personal reference range in order to compare results of measurements that are carried out in case of suspicion of disease based on the total variation including the within-person biological variation and the analytical variation of laboratory tests and the previous measurement results of test results, by using the results of laboratory tests measured in a period when persons feel healthy.

### BACKGROUND OF THE INVENTION

Persons who have their laboratory tests measured compare their results with a range lower and upper limits of which are determined in accordance with specific statistical rules called as reference ranges that do not belong to them and are mainly based on population. However, due to the fact that population-based reference ranges do not entirely reflect the individual's condition and physiological change, they may cause occurrence of medical errors in interpretation of patient results. Even though laboratory results of a person are included in population-based reference ranges, the person may not be healthy or the person may be healthy even though his/her laboratory results are out of population-based reference ranges. Therefore, although reference ranges being used today and based on population are obtained from persons with similar characteristics, they do not completely represent individuals and lead to occurrence of medical errors. For this reason, today there is need for solutions whereby medical errors resulting from reference ranges are avoided by means of use of a person's own data in interpretation of persons' laboratory results and different reference ranges particular to each individual are created.

The United States patent document no. US20160282361 discloses a method for carrying out mass spectrometric assays of peptides. The method in the said invention ensures interpretation of mass spectrometric tests of clinical biomarker peptides and addition of stable isotope labeled peptides without damaging the peptide proportioning. The said invention discloses personal reference values and personal reference ranges of patients whose mass spectrometric analysis assay of peptide is only carried out.

The Japanese patent document no. JP2018536846A, an application in the state of the art, discloses a method for personalizing biomarker signals and using personalized biomarker signals in medical procedures. In order to customize biomarker signals the said invention, a variation value is used in addition to intra-individual and inter-individual biological variation values wherein personalization is implemented, and analytical mean and standard deviation values of tests which are carried out in a period when the patient is healthy and they are used so as to determine a personal biomarker reference range by using a Bayesian method.

### SUMMARY OF THE INVENTION

An objective of the present invention is to realize a system which enables to determine personal reference ranges by using the total variation including the within-person biological variation and the analytical variation of laboratory tests and the previous measurement results of test results.

### DETAILED DESCRIPTION OF THE INVENTION

“A method used for determining personal reference range of medical laboratory tests” realized to fulfil the objective of the present invention is shown in the FIGURE attached, in which:

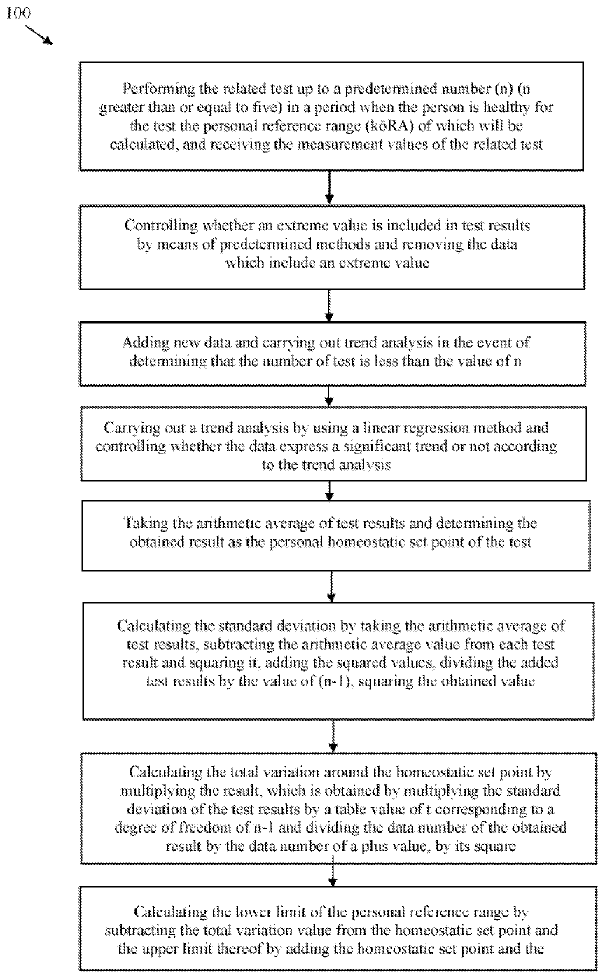

FIG. 1 is a view of steps in the flowchart of the inventive method used for determining personal reference range of medical laboratory tests

100. Method

Equivalents of the symbols used in the detailed description part are stated below.
TVₛₑₜ: Total variation around the homeostatic set point
TV: Total variation of the individual
HSN: Homeostatic set point
n: Measurement number (data number, test number) of the test
köRA: Personal reference range
X₁, X₂, . . . , Xₙ: Measurement results
Tα: Table value corresponding to n−1 degree of freedom

The inventive method (100) used for calculating personal reference ranges (köRA) of medical laboratory tests comprises steps of:
performing the related test up to a predetermined number (n) (n greater than or equal to five) in a period when the person is healthy for the test the personal reference range (köRA) of which will be calculated, and receiving the measurement values of the related test;
controlling whether an extreme value is included in test results by means of predetermined methods and removing the data which include an extreme value;
adding new data and carrying out trend analysis in the event of determining that the number of test is less than the value of n;
taking the arithmetic average of test results and determining the obtained result as the personal homeostatic set point of the test;
calculating the standard deviation by taking the arithmetic average of test results, subtracting the arithmetic average value from each test result and squaring it, adding the squared values, dividing the added test results by the value of (n−1), squaring the obtained value;
calculating the total variation around the homeostatic set point by multiplying the result, which is obtained by multiplying the standard deviation of the test results by a table value of t corresponding to a degree of freedom of n−1 and dividing the data number of the obtained result by the data number of a plus value, by its square;
calculating the lower limit of the personal reference range by subtracting the total variation value from the homeostatic set point and the upper limit thereof by adding the homeostatic set point and the total variation value.

In the inventive method (100), tests—the reference range of which are aimed to be determined—are selected and there are prerequisites such that the person has no disease symptom that will affect the test result and the person does not take medicine or medicines that will affect the test result during the measurement. Sample is taken from the person/persons, for whom it is aimed to determine the reference range for the related test, on different days and preferably at the same time of a day in n times (n≥5) and the related tests are measured in the samples taken. In the inventive method (100), measurement of the related test or tests are made in the samples which are taken on different days and at similar times of a 25 day (such as morning, midday, evening) in n times (n≥5). The received measurement results are used for determining the homeostatic set point (HSN) of the related test or tests.

In the inventive method (100), data are examined in terms of extreme values at first. If extreme values are available, the data is removed from the group and new data is added in the event that the number of data falls below the number of n. Statistical tests can be used preferably such as Dixon test for detection of extreme values. Following the analysis of extreme value, the data are evaluated in terms of trend. When the data are sorted by measurement dates, they should not exhibit a gradually increasing or decreasing trend. In the event of presence of an increasing or decreasing trend, the related data are not used and the personal reference ranges (köRA) cannot be calculated based on these data and new data are gathered. Linear regression method is used for trend analysis.

In the inventive method (100), HSN is calculated by using the data which are free of extreme values and do not exhibit a significant trend according to trend analysis. Arithmetic average of data are taken and the obtained values is accepted as the personal homeostatic set point of the related test.

$$HSN = \frac{{X}_{1} + {X}_{2} + {X}_{3} + … + {X}_{n}}{n}$$

In the inventive method (100), standard deviation is calculated by taking the arithmetic average of data, subtracting the arithmetic average value from each data (from the number one data to the last data) and squaring it, adding all squared data, dividing the added data by the value of (n−1), squaring the obtained data.

In the inventive method (100), the total variation is obtained by multiplying the standard deviation of data by a constant (Tα, table value of t corresponding to degree of freedom (n−1)) and the data number of the obtained result by the data number of a plus value, by its square.

$${TV}_{set} = {T}_{α} \times TV \times \sqrt{\frac{( n + 1 )}{n}}$$

In the inventive method (100), the lower limit of the personal reference range is calculated by subtracting the calculated total variation value from the homeostatic set point and the upper limit is calculated by adding it.

Personal reference range=Homeostatic set point±Total variation around the homeostatic set point

In the inventive system (1), information and approval are provided to users in accordance with principles of data confidentiality and it is acted within the scope of the Personal Data Protection Law (KVKK) at the steps of method (100).

Within these basic concepts; it is possible to develop various embodiments of the inventive method (100) used for determining personal reference range of medical laboratory tests; the invention cannot be limited to examples disclosed herein and it is essentially according to claims.

1. A method (100) used for calculating personal reference ranges (köRA) of medical laboratory tests; characterized in that it comprises steps of:
performing the related test up to a predetermined number (n) (n greater than or equal to five) in a period when the person is healthy for the test the personal reference range (köRA) of which will be calculated, and receiving the measurement values of the related test;
controlling whether an extreme value is included in test results by means of predetermined methods and removing the data which include an extreme value;
adding new data and carrying out trend analysis in the event of determining that the number of test is less than the value of n;
carrying out a trend analysis by using a linear regression method and controlling whether the data express a significant trend or not according to the trend analysis;
taking the arithmetic average of test results and determining the obtained result as the personal homeostatic set point of the test;
calculating the standard deviation by taking the arithmetic average of test results, subtracting the arithmetic average value from each test result and squaring it, adding the squared values, dividing the added test results by the value of (n−1), squaring the obtained value;
calculating the total variation around the homeostatic set point by multiplying the result, which is obtained by multiplying the standard deviation of the test results by a table value of t corresponding to a degree of freedom of n−1 and dividing the data number of the obtained result by the data number of a plus value, by its square;
calculating the lower limit of the personal reference range by subtracting the total variation value from the homeostatic set point and the upper limit thereof by adding the homeostatic set point and the total variation value.

2. A method (100) according to claim 1; characterized by the step of taking sample from the person/persons, for whom it is aimed to determine the reference range for the related test, on different days and preferably at the same time of day in n times (n≥5) and receiving the measurements obtained from the tests results.

3. A method (100) according to claim 1; characterized by the step of determining whether values that can be accepted as an extreme value are included in data or not by using statistical tests such as Dixon test, and removing the extreme values from the data group.

4. A method (100) according to claim 1; characterized by the step of not using data which exhibit increasing or decreasing trend by carrying out a trend analysis with a method such has linear regression.

5. A method (100) according to claim 1; characterized by the step of calculating the personal homeostatic set point of the related test by taking the arithmetic average of data which are free of extreme values and do not exhibit a significant trend according to trend analysis.

6. A method (1) according to claim 1; characterized by the step of calculating the total variation by multiplying the standard deviation of data by a constant (Tα, table value of t corresponding to degree of freedom (n−1)) and the data number of the obtained result by the data number of a plus value, by its square.

7. A method (1) according to claim 1; characterized by the step of calculating the lower limit of the personal reference range by subtracting the total variation value (TVset) from the homeostatic set point and the upper limit thereof by adding the homeostatic set point and the total variation value (TVset).
